(19) United States
(12) Patent Application Publication
(10) Pub. No.: US 2026/0207173 A1
(43) Pub. Date: Jul. 23, 2026

(54) APPARATUS AND METHOD FOR ESTIMATING A VELOCITY FIELD

(71) Applicant: E-SCOPICS, Aix-en-Provence (FR)

(72) Inventors: Francois MAURICE, Draguignan (FR); Adrien BESSON, Marseille (FR)

(21) Appl. No.: 19/139,050

(22) PCT Filed: Oct. 20, 2023
(86) PCT No.: PCT/IB2023/000639

§ 371 Date: Jun. 13, 2025

Related U.S. Application Data
(60) Provisional application No. 63/432,722, filed on Dec. 15, 2022.

(51) Int. Cl. A61B 8/00

(52) U.S. Cl. CPC A61B 8/485; A61B 8/5223; A61B 8/4483; A61B 8/488

(57) ABSTRACT

The invention concerns a method of determining 2D characteristics of a propagative displacement wave inside a patient, said method comprising: estimating a plurality of velocity maps of a target region over time, filtering (30) the propagative displacement wave within the plurality of velocity maps in order to obtain a filtered plurality of velocity maps, determining 2D characteristics including the propagation velocity and the propagation direction of the propagative displacement wave for each point of interest A within the plurality of velocity maps in order to obtain at least one map of 2D characteristics, said point of interest A having the same coordinates within the plurality of velocity maps.

### FIELD OF THE INVENTION

The present invention relates to a method for determining 2D (two-dimensional) characteristics of a propagative displacement wave inside a patient.

More specifically, the present invention relates to a method for measuring properties of a biological tissue of interest.

It applies, but not exclusively, to the measurement of viscoelasticity parameters of a liver or spleen of a human or an animal, this measurement being correlated with the amount of fibrosis present in the liver.

### BACKGROUND OF THE INVENTION

Shear wave elastography is a well-known technique used to measure viscoelastic properties (such as the stiffness) of a medium (such as tissues, organs, or other samples).

This technique consists in measuring the propagation velocity of a shear wave into the medium, this propagation velocity being directly related to the viscoelastic properties of the analyzed medium.

Shear-wave elastography induces medium displacements using either an ultrasound radiation force (Acoustic Radiation Force Impulse technique) or a mechanical vibrator (Vibration Controlled Transient Elastography VCTE™ technique, or the technique described in document WO2022084502).

In both cases the propagation direction of the shear wave front is known a priori, and the analysis of the induced shear wave propagation is performed in an analysis direction which is chosen to be a priori perpendicular to the assumed shear wave front.

Shear waves can also be naturally generated by living structures such as the heart, arteries, veins, and muscles.

### 1. Generation of a Shear Wave by Acoustic Radiation Force

In the case of a shear wave generation by acoustic radiation force, a shear wave is generated with an ARFI (Acoustic Radiation Force Impulse) transmitted as a push pulse: ultrasound energy is transmitted to a focal region of the medium in order to generate a shear wave, resulting in displacements of the medium around the focal region.

An ultrasound scanning step is implemented in order to track displacements of the medium over time in a region of interest (ROI) contained within the medium. Particularly, ultrasound compression waves are emitted at a fast rate after generating the shear wave (see document WO0055616). This ultrasound scanning step allows a succession of “echogenicity maps” of the medium to be obtained during the propagation of the shear wave. In particular, successive images of incremental displacements are determined by correlating successive echogenicity maps.

A processing step of these images of incremental displacements is then implemented in order to determine characteristics (e.g. propagation velocity along the analysis direction) of the shear wave. These characteristics are representative of specific viscoelastic properties of the medium.

However, the characteristics of the shear wave inferred from displacements may be biased if the analysis direction of the shear wave propagation differs from the actual propagation direction of the shear wave. Indeed, shear waves may undergo refraction or reflection because of e.g. inhomogeneities of the medium.

This involves that the processing step will not be performed along the correct direction, leading to an overestimation of the shear wave propagation velocity.

### 2. Generation of a Shear Wave by Mechanical Excitation

In the case of a shear wave generated by mechanical excitation (“Vibration Controlled Transient Elastography” technique (VCTE™) or the one described in WO 2022/084502), a shear wave source is mechanically coupled to the patient's skin. When actuating the shear wave source, the vibrations produced at a low frequency (typically between 30 Hz and 200 Hz) by the shear wave source onto the patient's skin allow a propagating shear wave to be generated within the medium. The shear-wave propagation direction is assumed to be perpendicular to the skin to shear wave source interface.

During a subsequent ultrasound scanning step, ultrasound shots are emitted to track displacements of the medium induced by this low frequency periodic vibration along one or several scan-lines that are supposed to be parallel to the shear-wave propagation direction, assumed to be known a priori. One dimensional displacements along the scan-lines are obtained. Such displacements are projections of the displacements caused by the shear wave propagation along the directions of the scan lines, at different points over the ROI, and at the same instant.

Considering the given assumption that the propagation direction of the shear wave is parallel to the scan-lines, a processing step of these scan-lines is implemented in order to determine shear wave propagation velocity. The shear wave propagation velocity is used to derive specific viscoelastic properties of the medium.

However, there are cases where the assumption relative to the propagation direction of the shear wave is incorrect, so that the shear wave propagation velocity which is determined is overestimated.

For instance, when imaging a patient's organ such as a liver, the shear wave source must be applied onto the patient's skin at a position located directly above and in between the patient's ribs. This involves the occurrence of undesirable effects (mechanical coupling effect and shear wave shading effect discussed in more details below) which modify the propagation direction of the shear wave. This modification in the propagation direction, if not taken into account during the processing step, leads to an overestimation of the shear wave propagation velocity.

### 3. Natural Generation of the Shear Wave

Finally, techniques—called “passive elastography”—rely on shear waves generated naturally by the body, e.g. using vibrations induced by heart beats.

In passive elastography, the propagation direction of the shear wave may be a priori unknown.

Hence, any solution such as the one described above regarding ARFI and transient elastography—wherein an assumption about the shear wave propagation direction is made prior to the implementation of the processing step—will lead to biased estimations.

### 4. Aim of the Present Invention

An object of the present invention is to propose a method of determining 2D characteristics of a propagative displacement wave allowing one to overcome at least one of the aforementioned drawbacks.

More specifically, an object of the present invention is to provide a method of determining 2D characteristics of a propagative displacement wave without any a-priori knowledge of the propagation direction of said propagative displacement wave.

### SUMMARY OF THE INVENTION

For this purpose, the invention proposes a method of determining 2D characteristics of a propagative displacement wave inside a patient, said method comprising:
estimating a plurality of velocity images/maps of a target region over time, said estimating phase including the steps consisting in:
   constructing a plurality of echogenicity maps by repeatedly implementing the following sub-steps for each echogenicity map:
      transmitting ultrasounds signals using an array of transducer elements including a group of transducer elements,
      receiving backscattered echo signals by the array of transducer elements, each transducer element allowing the acquisition of a respective temporal signal of a group of temporal signals corresponding to the amplitude of the received backscattered echo signals onto the array of transducer elements,
      constructing the echogenicity map from the group of temporal signals,
   estimating the plurality of velocity images/maps by correlating and processing the plurality of echogenicity maps,
filtering the propagative displacement wave within the plurality of velocity images/maps in order to obtain a filtered plurality of velocity images/maps,
determining 2D characteristics including the propagation velocity and the propagation direction of the propagative displacement wave for each point of interest A within the plurality of velocity images/maps in order to obtain at least one map of 2D characteristics, said point of interest A having the same coordinates within the plurality of velocity images/maps by:
   associating at least two points B, C within the plurality of velocity images/maps such that points A, B, C are non-collinear, said at least two points B, C having the same coordinates within the plurality of velocity images/maps,
   extracting a temporal propagative displacement signal from the filtered plurality of velocity images/maps for each of the three points A, B, C (the point of interest A and the at least two points B, C),
   estimating first and second apparent times of flight (in particular: a “longitudinal” apparent time of flight between points A and B, and a “transversal” apparent time of flight between points A and C) of the propagative displacement wave between the three points A, B and C (the point of interest A and the at least two points B, C) using the temporal propagative displacement signals associated to the three points A, B, C (the point of interest A and the at least two points B, C) by:
      intercorrelating the temporal propagative displacement signals associated to points A, B (the point of interest A and one (B) of the at least two points B, C) for estimating the first apparent time of flight (longitudinal apparent time of flight) of the propagative displacement wave from point A to B (from the point of interest A to the one (B) of the at least two points B, C),
      intercorrelating the temporal propagative displacement signals associated to points A, C (the point of interest A and another (C) of the at least two points B, C) for estimating the second apparent time of flight (transversal apparent time of flight) of the propagative displacement wave from point A to C (from the point of interest A to the another (C) of the at least two points B, C),
   determining propagation direction and propagation velocity of the propagative displacement wave based on the first and second apparent times of flight.

In the context of the present invention, the expression “propagative displacement wave” refers to a shear wave generated either naturally (i.e. passive elastography) or artificially (i.e. transient elastography).

Preferred—but non-limiting—aspects of the method according to the invention are the following:
the method can further comprise a step of estimating a stiffness of a tissue contained within the target region using the map of 2D characteristics;
the step of estimating the stiffness of the tissue can include:
   extracting velocities of the propagative displacement wave for a subset of points from the map of 2D characteristic, said subset of points being representative of the tissue contained within the target region, and
   computing a median, or an average of extracted velocities of the propagative displacement wave for the subset of points,
   estimating the stiffness from said computed median or average;
step of estimating the stiffness of the tissue can include:
   computing a map of stiffness of the region of interest from the map of 2D characteristics,
   extracting stiffnesses of a subset of points from the map of stiffness, said subset of points being representative of the tissue contained within the target region, and
   computing a median, or an average of extracted stiffnesses of the subset of points;
the method can further comprise a step of determining a quality factor relative to an average propagative displacement direction, said step of determining the quality factor including:
   extracting directions of the propagative displacement wave for a subset of points from the map of 2D characteristics,
   computing an average direction of propagation of the propagative displacement wave from the extracted directions of the propagative displacement wave for the subset of points, and
   comparing the average direction of propagation of the propagative displacement wave to a predefined range of directions to obtain a comparative data, and
   deriving the quality factor according to the comparative data;
the method can further comprise a step of determining a coefficient representative of the pressure applied by the array of transducers onto the patient, said step of determining the coefficient including the sub-steps of:
   computing a curvature of the wave front of the propagative displacement wave based on the map of 2D characteristics,
   if the computed curvature is concave corresponding to a convergent propagative displacement wave, assigning to the coefficient a value representative of a light pressure,
   if the computed curvature is convex corresponding to a diverging propagative displacement wave, assign to the coefficient a value representative of a strong pressure higher than the light pressure;
the method can further comprise a step of determining a parameter representative of the quality of the propagative 2D displacement estimation, said step of determining the parameter including the sub-steps of:
   computing a variance of the transversal apparent time of flight (variance of the time of flight between points A and C) which is representative a spatial variance of the direction of the propagative displacement wave,
   comparing said quantity to a predefined threshold and determining the parameter according to the result of said comparison sub-step;
the step of estimating the plurality of velocity images/maps by correlating and processing the plurality of complex echogenicity maps includes:
   cross-correlating the plurality of complex echogenicity maps pair by pair with a certain temporal lag to obtain a plurality of phase-shift maps
   deriving the plurality of velocity maps from related plurality of phase-shift maps;
the sub-step of constructing the echogenicity map from the group of temporal signals comprises the resolution of an inverse problem to produce said echogenicity map.

The invention also describes a method for estimating a shear wave velocity V within a biological tissue, the method comprising:
detecting a shear wave that has been generated in the biological tissue by a shear wave source, said shear wave being locally characterized by a shear wave front propagating along a propagation direction,
computing temporal displacement signals representative of the propagation of the shear wave front within a region of interest of the biological tissue,
determining times of maximum intercorrelation of the shear wave temporal displacements between at least three non-colinear points located at different locations within the region of interest:
   a first point and a second point extending along a first line within the region of interest (assumed to be roughly parallel to the direction of the shear wave front),
   the first point and a third point extending along a second line within the region of interest (assumed to be roughly perpendicular to the propagation direction of the shear wave), and
determining the local shear wave velocity V based on said times.

Preferred—but non-limiting—aspects of the method according to the invention are the following:
the step of determining the shear wave velocity comprises the following sub-steps:
   computing a first apparent shear wave front velocity V₁ along a first line assumed to be roughly parallel to the propagation direction of the shear wave,
   computing a second apparent shear wave front velocity V₂ along a second line assumed to be roughly perpendicular to the propagation direction of the shear wave,
the step of determining the local shear wave velocity V comprises a sub-step of estimating said local shear wave velocity V based on the first and second shear wave front velocities V₁, V₂;
advantageously:
   for the computing of the first apparent shear wave front velocity V₁, the method comprises:
      selecting first and second points A, B located at different locations along the first line, the first point A being closer from the array of transducers than the second point B, and deducing a first distance between the first and second points A, B,
      determining, using the temporal propagative displacement signals, a first apparent travel time of the shear wave front between the first and second points A, B,
      deriving the first apparent shear wave front velocity V₁ by determining a ratio between the first distance divided by the first travel time,
   for the computing of the second apparent shear wave front velocity:
      selecting a third point C located along the second line, and deducing a second distance between the first and third points A, C,
      determining, using the temporal prapagative displacement signals, a second apparent travel time of the shear wave front between the first and third points A, C,
      deriving the second apparent shear wave front velocity V₂ by determining a ratio between the second distance divided by the second travel time.

The invention also concerns a process for assisting a user in positioning a probe and controlling the pressure onto a surface of an anatomical structure to be imaged, the probe comprising an array of transducers (T₁-Tₙ) for imaging the anatomical structure, and at least one vibrator for generating a shear wave through the anatomical structure, said process comprising implementing the method described above.

### BRIEF DESCRIPTION OF THE DRAWINGS

The present invention may be more completely understood in consideration of the following detailed description of various embodiments in connection with the accompanying drawings, in which:

FIG. 1 is a schematic view of phases implemented in a method for determining 2D characteristics (propagation velocity and propagation direction) of a propagative displacement wave inside a patient,

FIG. 2 is a schematic representation of a processing assembly for implementing the method illustrated on FIG. 1,

FIG. 3 is a schematic representation of steps implemented within a tracking phase illustrated on FIG. 1,

FIG. 4 is a schematic representation of steps implemented within a processing phase illustrated on FIG. 1,

FIG. 5 is a schematic representation illustrating the radiation pattern of a shear wave generated by a point source,

FIG. 6 is a schematic representation illustrating an ultrasonic pulse elastography probe implemented in Fibroscan® system,

FIG. 7 is a schematic representation illustrating the radiation pattern of shear waves generated by Fibroscan® system,

FIG. 8 is a schematic representation illustrating an ultrasonic pulse elastography probe implemented in Hepatoscope,

FIG. 9 is a schematic representation illustrating shear waves generation areas on the ultrasonic probe of Hepatoscope,

FIGS. 10a and 10b are schematic representations illustrating a shear wave shading effect in a plane perpendicular to ribs of a patient when using Fibroscan® and Hepatoscope, respectively,

FIGS. 11a and 11b are schematic representations illustrating the shear wave shading effect in a plane parallel to the patient's ribs when using Fibroscan® and Hepatoscope, respectively,

FIGS. 12a and 12b are schematic representations illustrating a mechanical coupling effect in a plane perpendicular to the patient's ribs, when using Fibroscan® and Hepatoscope, respectively,

FIGS. 13a and 13b are schematic representations illustrating the mechanical coupling effect in a plane parallel to the patient's ribs when using Fibroscan® and Hepatoscope, respectively,

FIG. 14 is a geometric scheme illustrating a shear wave front analysis,

FIG. 15 is a schematic scheme illustrating the extraction of a temporal propagative displacement signal from filtered velocity maps.

### DETAILED DESCRIPTION OF THE INVENTION

Different examples of the method and apparatus according to the invention will now be described with reference to the figures. In these different figures, equivalent elements are designated by the same numerical reference.

### 1. Method for Determining 2D Characteristics of a Propagative Displacement Wave

### 1.1. Generalities

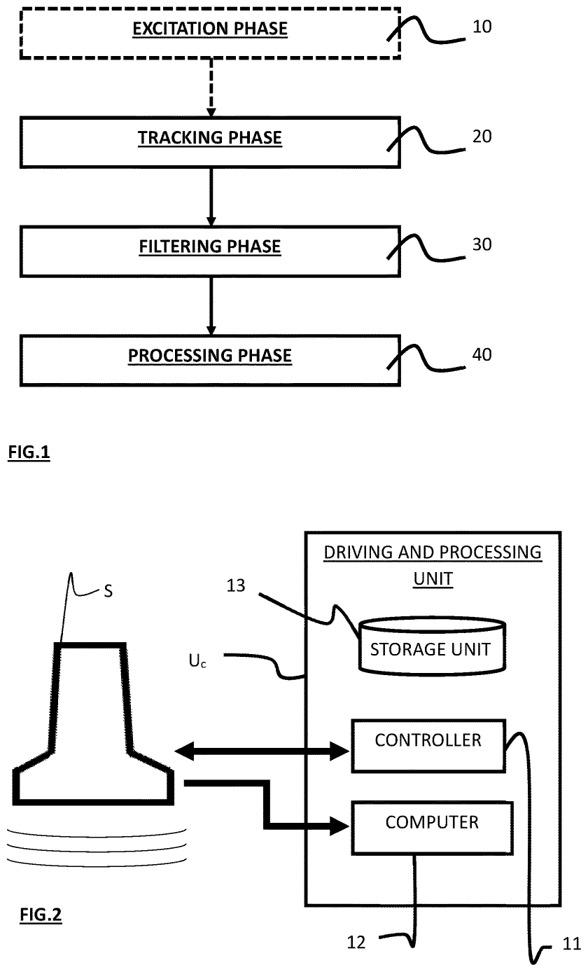

Referring to FIG. 1, the steps of a method configured to determine 2D characteristics of a propagative displacement wave propagating inside a region of interest (ROI) of a patient are illustrated.

As will be described in more details below, the 2D characteristics of the propagative displacement wave may be:
the propagation velocity of the propagative displacement wave,
the propagation direction of the propagative displacement wave, or
more generally any 2D representation (cartesian, polar . . . ) of the displacement wave velocity vector.

In the context of the present invention, the propagative displacement wave can be a shear wave generated:
either naturally (i.e., passive elastography), the propagative displacement wave being generated by an organ such as the heart during a cardiac cycle, or
artificially (i.e., transient elastography), the propagative displacement wave being generated by an external source, such as an inertial vibration exciter of the type described in WO 2022/084502.

The method comprises the following phases:
a) an optional excitation phase 10, wherein the propagative displacement wave is generated
b) a tracking phase 20 wherein the displacements, within the ROI, of the medium partly induced by the propagative displacement wave are tracked; this tracking phase allows a plurality of velocity maps of the ROI to be estimated over time,
c) a filtering phase 30 for isolating the propagative displacement wave within the plurality of velocity maps,
d) a processing phase 40 during which the 2D characteristics of the propagative displacement wave are determined from the plurality of velocity maps.

In the following, the analysis method will be described with reference to the processing of the data acquired using an ultrasound probe allowing:
to generate at least one low-frequency elastic wave—called “propagative displacement wave”—in the medium, and
simultaneously with the generation of the low-frequency elastic wave:
   to emit high-frequency ultrasonic waves, and
   to receive acoustic echoes due to the reflections of high-frequency ultrasonic waves in the medium,
      in order to observe the propagation of the low frequency elastic wave in the medium.

The skilled person will nevertheless understand that the analysis method can be implemented in the context of passive elastography techniques—in which the shear waves are generated naturally by the body. In this case, the ultrasound probe comprises an array of transducer elements for the emission of high-frequency ultrasonic waves (1-20 MHz) and the reception of acoustic echoes in order to observe the propagation of one (or more) low frequency elastic wave(s) generated naturally by the body.

### 1.2. Excitation Phase (Optional)

With reference to FIG. 2, a processing assembly for implementing the method according to FIG. 1 has been illustrated.

This processing assembly includes:
a signal acquisition probe S, and
a driving and processing unit Uc for:
   controlling the probe S, and
   processing the signals acquired by the probe S.

The probe S includes: an array of transducer elements for the emission of ultrasonic waves and the reception of acoustic echoes, and an exciter for generating the shear wave. The exciter may be an inertial vibration exciter of the type described in document WO 2022/084502. A fixed part of the exciter is mechanically attached to the array of transducer elements to transmit the vibrations to the probe in order to mechanically produce the propagative displacement wave.

The driving and processing unit Uc is connected to the probe S by wired or wireless communication means. The driving and processing unit Uc is configured to control the transducer elements of the probe S, and to process the data acquired by the transducers of the probe S. The driving and processing unit Uc is further configured to activate the inertial vibration exciter for the generation of one (or more) propagative displacement wave(s) in the medium. More specifically, the driving and processing unit Uc is configured: to control the inertial vibration exciter to produce a vibration allowing the generation of a propagative displacement wave into the medium, to command the transmission by the transducer elements of high-frequency ultrasonic waves in the medium, to command the reception by the transducer elements of the echoes reflected by the medium, to convert said echoes in electrical reception signals, and to process the reception signals.

Transmission and reception electronics are located between the driving and processing unit Uc and the probe S and provide the necessary signal generation, amplification, digitization and transmission between probe and Uc. This hardware can be physically located close to Uc, and connected to the probe using tiny coax cables carrying analog signals, or can be situated in the probe handle, in which case the communication between probe and Uc is using a purely digital link.

The driving and processing unit Uc can be composed of one or more distinct physical entities, possibly remote from the probe S. For instance, the driving and processing unit Uc can be composed of:
one (or several) controler(s) 11, such as a Smartphone, and/or an electronic tablet (such as an IPAD®) and/or a Personal Digital Assistant (PDA), or chips on an electronic board (e.g. microcontroller, FPGA) or may be of any other type known to the skilled in the art, and
one (or several) computer(s) 12 (a beamformer, a processor, etc.), such as a personal computer and/or a workstation, etc.
one (or several) storage unit 13 including at least one memory such as a Random Access Memory (RAM) and/or a Read Only Memory (ROM) and/or an USB key, a cloud storage, etc. The storage unit may be part of the controller 11, or of the computer 12.

In addition to the storage of the acquired/processed data, the storage unit 13 allows programming code instructions to be stored, said programming code instructions being intended to execute the phases of the analysis method illustrated on FIG. 1.

The operating principle of the processing assembly illustrated on FIG. 2 is described below.

In order to implement the excitation phase 10, a user put the probe S into contact with the patient's skin. For instance, if the user wishes to analyze properties of the patient's liver, the array of transducer elements is put in contact with the patient's skin in the right ribs region, so that the array of transducer elements extends substantially between two adjacent ribs.

The driving and processing unit Uc actuates the exciter in order to produce vibrations. The vibrations generate the propagative displacement wave inside the patient.

### 1.3. Tracking Phase

During the tracking phase 20, the displacements of the medium induced by the propagation of the propagative displacement wave are measured within a ROI by pulse-echo ultrasound (e.g. tissue Doppler). Particularly, a plurality of velocity maps of the ROI is estimated over time during the tracking phase. These velocity maps correspond to “displacement images”, and include information representative of the propagation of the propagative displacement wave in the ROI.

In order to implement the tracking phase, the driving and processing unit Uc controls the emission of a succession of ultrasonic waves (each ultrasonic wave having a center frequency comprised between 1 MHz and 20 MHz for example) by the array of transducer elements of the probe S.

These ultrasonic waves penetrate into the medium and are reflected on scattering particles contained in said medium-such as for example collagen particles-which makes it possible to follow displacements of such scattering particles within the medium.

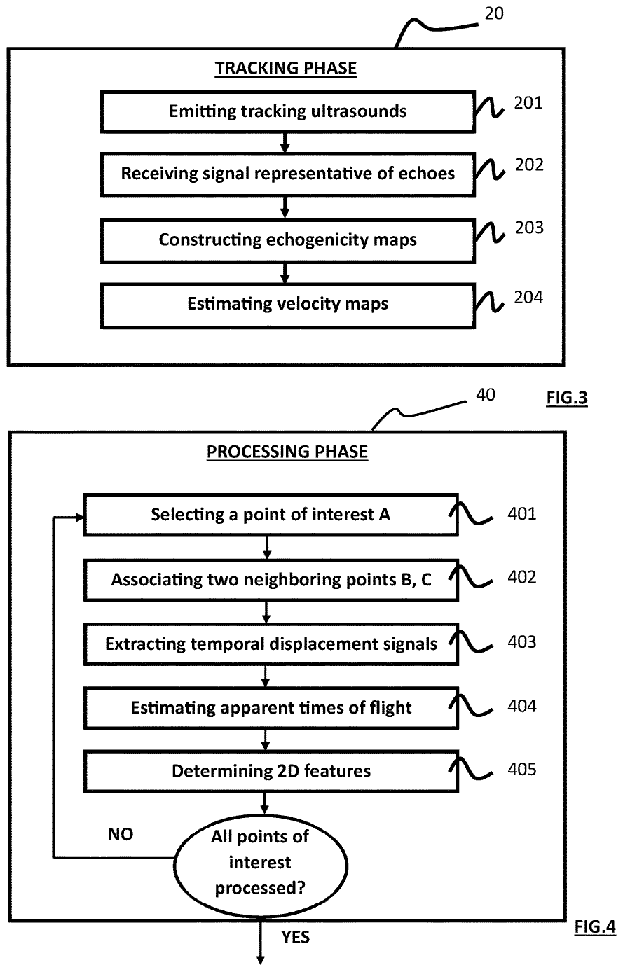

In particular, and referring to FIG. 3, the tracking phase 20 comprises the following sub-steps:
the driving and processing unit Uc controls the transmission by the array of transducer elements, of a succession of ultrasonic waves in the medium at a rate between 10 and 10,000 shots per second (step 201),
the driving and processing unit Uc controls the reception by the array of transducer elements, and the recording (in real time) in the storage unit 13 of time-dependent acoustic signals received by the transducers of the array, said time-dependent acoustic signals being representative of the echoes generated by the ultrasonic waves interacting with the scattering particles of the ROI (step 202),
the driving and processing unit Uc:
   constructs a plurality of complex echogenicity maps of the ROI using the time-dependent acoustic signals (step 203), and
   estimate the plurality of velocity maps of the ROI over time based on the plurality of complex echogenicity maps (step 204).

More precisely, the sub-step of constructing the plurality of echogenicity maps consists in determining images of the ROI contained in the field of tracking (i.e., the zone insonified by the ultrasonic waves) by solving an inverse problem which may be reduced to conventional “beamforming”. Each echogenicity map results from the compound processing of one or several consecutive shots.

During the subsequent sub-step of estimating the plurality of velocity maps, these successive images of the ROI are processed, by filtering and correlation and in particular by cross-correlation:
either two by two with a certain lag (temporal),
or with a reference image.

### 1.4. Filtering Phase

As mentioned above, the velocity maps are representative of the propagation of the propagative displacement wave in the ROI. However, the velocity maps may also include components, which can significantly interfere with the propagative displacement wave.

The filtering phase 30 isolates the part of the displacements due to the propagation of the propagative displacement wave from other artefact components (e.g., static deformation, compressional waves, out-of-plane shear waves or reflected waves).

The filtering phase can be based on different techniques known in the art. For example, the filtering phase can be implemented using the solution disclosed in document entitled “PROCEDE D'ANALYSE D'UN MILIEU PERMETTANT DE REDUIRE LES EFFETS D'ARTEFACTS DUS A DES DEFORMATION STATIQUES DANS LE MILIEU” whose filing number is FR2210011.

The filtering phase can also be based on temporal filtering (widely used in harmonic elastography as it permits to band-pass filter the velocity maps around the frequency of interest), or on spatial filtering, or on spatiotemporal filtering. An example of such a technique is disclosed in the article of Manduca et al. (A. Manduca, D. S. Lake, S. A. Kruse, and R. L. Ehman, “Spatio-temporal directional filtering for improved inversion of MR elastography images”, Med. Image Anal., vol. 7, no. 4, pp. 465-473, December 2003).

In any case, the filtering phase allows one to obtain a plurality of filtered velocity maps wherein some unwanted components have been removed.

### 1.5. Processing Phase

Once filtered, the plurality of filtered velocity maps is processed in order to determine 2D characteristics of the propagative displacement wave for each point of interest within the plurality of filtered velocity maps of the ROI. The 2D characteristics of the propagative displacement wave can consist in:
the propagation velocity of the propagative displacement wave, and
the propagation direction of the propagative displacement wave.

These 2D characteristics of the propagative displacement wave are estimated for each point of interest within the plurality of velocity maps. This allows one to obtain a map of 2D characteristics of the propagative displacement wave.

In the following, the processing phase will be described for a single point of interest A of the ROI, being understood that the different steps disclosed below can be repeated for any point of interest (A′, A″, etc.) of the ROI.

### 1.5.1. Association of Neighboring Points

The processing phase comprises associating two (or more than two) neighboring points B, C (step 402) to the point of interest A.

The two neighboring points B, C are selected such that the three points A, B, C are non-collinear.

The skilled person will appreciate that the points A, B, C have the same coordinates within all the filtered velocity maps, since the plurality of filtered velocity maps illustrates the same ROI.

### 1.5.2. Extraction of Temporal Displacement Signals

The processing phase comprises a step of extraction (step 403) of temporal propagative displacement signals S_{A}, S_{B}, S_{C} for points A, B and C from the filtered plurality of velocity images/maps.

It is reminded that each filtered velocity map:
represents the velocity of the propagative displacement wave at each point of the ROI, and
corresponds to a respective time t during the propagation of said propagative displacement wave.

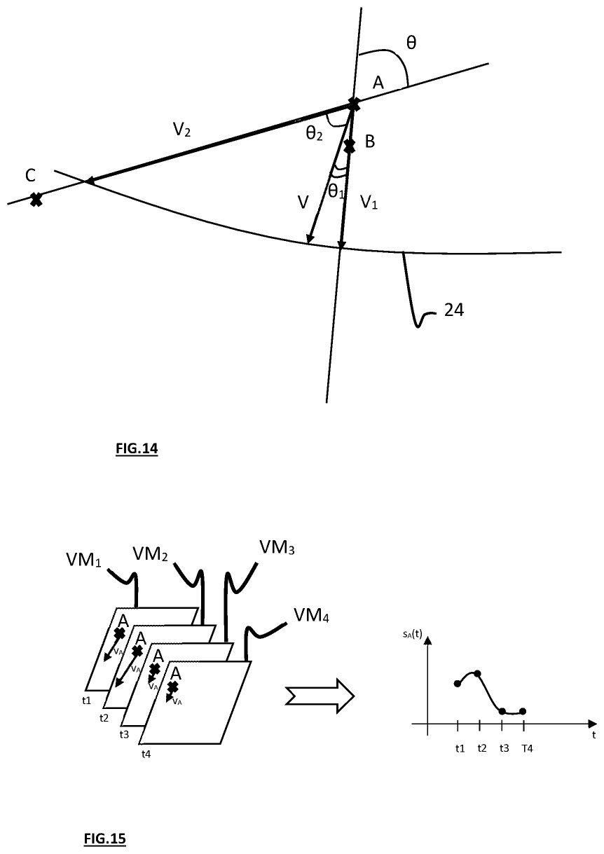

As illustrated on FIG. 15 for point A, the determination of the temporal propagative displacement signal S_{A} consists in expressing the velocities V_{A} at point A estimated at different time instants t₁, t₂, t₃, t₄ in the velocity maps VM₁, VM₂, VM₃, VM₄.

The temporal propagative displacement signals S_{B}, S_{C} are obtained in a same manner by extracting the velocities V_{B}, V_{C} contained in the plurality of velocity maps VM₁, VM₂, VM₃, VM₄, each velocity maps corresponding to a respective time instant t₁, t₂, t₃, t₄.

### 1.5.3. Estimation of Apparent Times of Flight

After obtaining the temporal propagative displacement signals S_{A}, S_{B}, S_{C} the processing phase comprises a step of apparent times-of-flight estimation (step 404) from the temporal propagative displacement signals S_{A}, S_{B}, S_{C}. This apparent time-of-flight estimation step determines the travel time of the propagative displacement wave between points A and B on the one end, and between points A and C on the other end.

Each temporal propagative displacement signal S_{A}, S_{B}, S_{C} is representative of the displacement of the propagative displacement wave at a respective point A, B, C of the ROI.

In other words, the temporal propagative displacement signals S_{A}, S_{B}, S_{C} are representative of the relative change in position of the scattering particles within the medium induced by the propagative displacement wave as it travels through the medium.

Since points A, B and C are distinct from one another, and since the propagative displacement wave is assumed to be invariant as it travels through the medium, the temporal propagative displacement signals S_{A}, S_{B}, S_{C} are identical but shifted in time (for instance, the propagative displacement wave reaches point A before reaching point B or point C).

In order to determine the apparent time-of-flight of the propagative wave between points A and B (longitudinal apparent time-of-flight), the temporal propagative displacement signals S_{A} and S_{B} are intercorrelated to measure a similarity of signals S_{A} and S_{B} as a function of the time shift of one (for instance S_{A}) relative to the other (for instance S_{B}). This allows one to assess the time delay between signals S_{A} and S_{B}, said time delay corresponding to the longitudinal apparent time-of-flight of the propagative displacement wave between points A and B.

In order to determine the apparent time of flight of the propagative wave between points A and C (transversal apparent time-of-flight), the temporal propagative displacement signals S_{A} and S_{C} are intercorrelated to measure a similarity of said signals S_{A} and S_{C} as a function of the time shift of one (for instance S_{A}) relative to the other (for instance S_{C}). This allows one to assess the time delay between signals S_{A} and S_{C}, said time delay corresponding to the transversal apparent time-of-flight of the propagative displacement wave between points A and C.

### 1.5.4. Determination of the 2D Features of the Propagative Displacement Wave

As mentioned above, neighboring points B and C are selected such that:
segment [A, B] is assumed to be roughly parallel to the direction of propagation of the propagative displacement wave,
segment [A, C] is assumed to be roughly perpendicular to the direction of propagation of the propagative displacement wave,
the angle θ (or BÂC) between B, A and C is assumed to be roughly equal to π/2 (i.e., segment [A, B] is perpendicular to segment [B, C]).

Based on the longitudinal and transversal apparent times of flight, the determination of 2D features—such as the propagation velocity and the propagation direction—of the propagative displacement wave can be implemented (step 405).

### 1.5.4.1. Computation of Longitudinal and Transversal Apparent Velocities

A longitudinal apparent shear wave front velocity V₁ between points A and B can be determined by computing a ratio between:
the distance separating points A and B (called “first distance” hereafter), divided by
the longitudinal apparent time of flight between points A and B.

Indeed, the first distance is known since positions of points A and B are known (from the association of neighboring point B during the association step), and the longitudinal apparent time of flight has been calculated in step 1.5.3.

In a similar manner, a transversal apparent shear wave front velocity V₂ between points A and C can be derived by computing a ratio between:
a second distance separating points A and C (a priori known from the association step wherein point C is associated to point A), divided by
the transversal apparent time of flight between points A and C.

### 1.5.4.2. Computation of the Real Velocity and Direction of the Propagative Displacement Wave

Using the computed longitudinal and transversal apparent shear wave front velocities V₁, V₂:
the real shear wave front velocity V, and
   the real propagation direction
      of the propagative displacement wave can be derived. See Section 3.1.8 for details.

### 1.5.4.3. Summary

The above-described method allows one to determine:
the real propagation velocity of the propagative displacement wave, and
the real propagation direction of the propagative displacement wave,
   by selecting neighboring points B and C of a point of interest A (said points B and C being selected such that (AB) and (AC) are roughly perpendicular), by estimating longitudinal and transversal apparent times of flight of the propagative wave between points A, B and C (using temporal propagative displacement signals S_{A}, S_{B}, S_{C}), and by deriving longitudinal and transversal apparent velocities V₁, V₂ of the propagative wave between points A, B and C, and by deducing the real velocity and direction of the propagative displacement wave using the longitudinal and transversal apparent velocities V₁, V₂.

The processing phase disclosed above is implemented for a plurality of points of interest (A, A′, A″, etc.). This allows one to obtain a map of 2D characteristics of the propagative displacement wave within the ROI.

This map of 2D characteristics of the propagative displacement wave can then be used for a plurality of applications.

### 2. Applications

### 2.1. Stiffness Estimation

The map of 2D characteristics of the propagative displacement wave can for instance be used in order to determine an estimate of the stiffness of the medium within the ROI. Indeed, the propagation velocity (speed) of the propagative displacement wave varies according to the stiffness of the target region. The propagative displacement wave has a significantly higher velocity (typically up to 5 m/s) in a fibrotic liver than in a healthy liver (typically 1 m/s).

To determine the stiffness of a tissue located within the region of interest, the method can comprise a step of estimating the stiffness of the target tissue using the map of 2D characteristics (and more particularly the velocity information contained within said map).

The estimation of the stiffness of a given subset of points (corresponding to the target tissue) within the region of interest can include the sub-steps of:
computing a median, or an average of the propagation velocities for said subset of points, and
estimating the stiffness from said median or average of propagation velocities, for instance using simple relationship between stiffness and velocity (e.g. E=3*c² valid for quasi-incompressible media like biological tissues, E being the stiffness and c being the velocity).

Alternatively, the estimation of the stiffness of a given subset of points (corresponding to the target tissue) within the region of interest can include the sub-steps of:
computing a map of stiffness from the map of 2D characteristics (and more particularly the velocity information contained within said map), for instance using simple relationships between stiffness and velocity (e.g. E=3*c² valid for quasi-incompressible media like biological tissues, E being the stiffness and c being the velocity), and then
computing a median, or an average of stiffness values of the subset of points from the map of stiffness.

### 2.2. Determination of a Quality Factor Relative to the Orientation and Position of the Probe

The map(s) of 2D characteristics of the propagative displacement wave can also be used in order to advise a user whether the probe is correctly oriented and positioned onto the patient.

To this end, the method can comprise a step of determining a factor representative of a quality of the orientation or the position of the probe onto the patient. Such a step of determining the quality factor can include the sub-steps of determining an average shear wave propagation direction (or angle) for a subset of points of the map of 2D characteristics, of comparing said average shear wave propagation to a preferred angle range (which is predefined), and of providing the quality factor for probe orientation and probe position based on the result of said comparison:
if the average shear wave propagation angle lies within the predefined range, the orientation or position of the probe is considered as being correct and the quality factor indicates to the user that the probe is correctly oriented or positioned,
if the average shear wave propagation angle is outside the predefined range, the orientation or position of the probe is considered as being incorrect, and the quality factor indicates to the user that the probe is not correctly oriented or positioned; the user can modify the orientation or the position of the probe.
   2.3. Determination of a Coefficient Representative of the Pressure Applied by the Probe onto the Patient

In addition, the map of 2D characteristics of the propagative displacement wave can be used in order to provide the user with information relative to the pressure applied by the probe onto the patient's skin in the context of transient elastography.

To this end, the method can comprise a step of determining a coefficient representative of the pressure applied by the probe onto the patient, said determining step including the sub-steps of:
computing a curvature of the wave front of the propagative displacement wave based on the map of 2D characteristics,
comparing the computed curvature to a reference information to determine whether the curvature is concave or convex,
if the computed curvature is concave, assigning to the coefficient a value representative of a light pressure,
if the computed curvature is convex, assigning to the coefficient a value representative of a strong pressure.

### 2.4. Determination of a Parameter Relative to the Quality of Contact Between the Probe and the Patient

Additionally, the map of 2D characteristics of the propagative displacement wave can be used in order to advise a user whether the contact between the probe and the patient's skin is of good quality or not.

To this end, the method can comprise a step of determining a parameter representative of the quality of contact between the probe the patient's skin, said determining step including the sub-steps of estimating a variance of the directions of the propagative displacement wave from the map of 2D characteristics, and determining the value of said parameter according to the determined variance. An example embodiment could be to compare the determined variance to a predefined threshold. If the variance exceeds such a threshold, the quality of contact between the probe and the patient is considered as low.

In another embodiment we can compute the variance of the transversal time of flight (corresponding to the time of flight between points A and C), which is a quantity representative of the spatial variance of the direction of the propagative displacement wave.

### 3. Theory Relative to the Invention

### 3.1. Detailed Theorical Description of the Invention

### 3.1.1. General Problem, and Proposed Solution

In ultrasound elastography, tissue displacements could be induced using:
a mechanical vibrator (Fibroscan VCTE technique),
ultrasound radiation force (ARFI, Supersonic Shear wave elastography),
natural vibrations of the body (e.g. heart beats).

In both cases, a major assumption is that the displacement direction is known, and the analysis of the induced shear wave propagation is performed in a direction which is chosen to be a priori perpendicular to the shear wave front.

There are cases where this a priori assumption is false:
In transient elastography, i.e. when the shear wave propagation is induced by an external vibration, if there is a mechanical coupling between the elastography probe (shear wave source) and the ribs, the shear waves may originate from the rib edges. In this case the propagation direction may be angled with respect to the probe symmetry axis, leading to an overestimation of liver stiffness,
In ARFI or supersonic shear wave imaging, if the medium is not homogeneous, the shear waves may undergo refraction and reflection. Hence, they will not be analyzed along their propagation direction, leading again to stiffness overestimation,
In passive elastography (where shear waves are generated using natural vibrations of the body), the propagation direction of the shear waves is a priori unknown, and again a shear wave analysis using a chosen a-priori propagation direction will lead to biased results.

We propose to solve the problems described above by performing a 2D analysis of the propagation of generated shear waves, allowing us to:
estimate the shear wave propagation direction in a 2D imaging plane,
use the true shear wave propagation direction to estimate a correct, non-overestimated stiffness value,
help the operator in positioning and orienting the probe as well as applying the right amount of pressure onto the patient, based on shear wave propagation direction measured in the 2D imaging plane in the case of liver elastography.

### 3.1.2. Doppler Processing for Velocity Maps Estimation

Doppler processing aims at extracting displacements of structures such as blood and tissues from successive transmissions of ultrasound (US) beams. In US imaging, displacement extraction is performed by analyzing variations in the plurality of echogenicity maps, which could be either in-phase quadrature (IQ) or radio-frequency (RF) beamformed maps (i.e. echogenicity maps), along the slow-time scale. Hence, all the techniques first start with the transmission/reception and beamforming of a set of IQ/RF images in order to obtain echogenicity maps, denoted as slow time samples. Such images are usually acquired with a constant rate denoted as the pulse-repetition frequency (PRF).

In order to extract Doppler frequency shifts from RF or IQ image slow-time samples, many techniques have been developed in the literature. Nevertheless, the different techniques share the common idea of tracking the translation of recorded backscattered signals between consecutive pulses.

The different methods differ in the way such a motion is tracked which mainly depends on the nature of the received backscattered echoes (RF or IQ, narrowband or broadband).

### 3.1.2.1. Phase-Based Estimators

A well-known technique consists in computing the phase-shift using the lag-1 temporal autocorrelation usually denoted as Kasai autocorrelation (or 1D autocorrelation), as described by Angelsen (B. A. Angelsen, “Instantaneous frequency, mean frequency, and variance of mean frequency estimators for ultrasonic blood velocity Doppler signals”, IEEE Trans. Biomed. Eng., vol. 28, no. 11, pp. 733-741, November 1981), Kasai and Nagekawa (C. Kasai and K. Namekawa, “Real-time two-dimensional blood flow imaging using an autocorrelation technique”, in IEEE 1985 Ultrasonics Symposium, San Francisco, CA, USA, 1985. doi: 10.1109/ultsym.1985.198654). The Kasai 1D autocorrelation method is briefly recalled below.

Formally, consider that we have access to NEL (ensemble length) consecutive slow-time samples, each slow-time sample being a beamformed image (i.e. an echogenicity map) on a grid composed of Nr×N_{θ} pixels. Slow-time samples are acquired at a time interval T_{P}, the pulse repetition period, with T_{P}=1/f_{P} where f_{P} is the pulse repetition frequency (PRF). The Doppler phase shift ΔΨ∈ [symbol] ^{N}^{ᵣ}^{×N}^{_{θ}} could be estimated from the following formula:

$$ΔΦ = {∠R}_{1} with {R}_{1} = {\sum }_{k = 1}^{{N}_{EL} - 1} {w}_{k + 1} {γ}_{k + 1} {w}_{k} {γ}_{k}^{*} ,$$

where γₖ are the slow time samples and ωₖ are windowing coefficients.

Note that the ensemble length can be down to 2 consecutive frames. The estimated phase shift and the Doppler frequency shift are linked by the following equation:

$$2 πδ {f}_{p} \times {T}_{P} = ΔΦ$$

In addition, we can relate the velocity to the Doppler frequency shift as

$$v = - c \frac{δ {f}_{p}}{2 {f}_{0}} = - c \frac{ΔΦ}{4 π {T}_{P} {f}_{0}} .$$

where c corresponds to the mean speed of sound and f₀ is the demodulation frequency.

Hence, we can intuit that there exists a maximal velocity associated with the given PRF corresponding to a maximal phase shift between consecutive signals which can be derived from the above equation as:

$${v}_{max} = \frac{π c}{4 π {T}_{P} {f}_{0}} = \frac{λ}{4 {T}_{P}} .$$

Regarding the Loupas estimator, the main difference with the Kasai 1D correlator is that the mean frequency used to extract the velocity from the Doppler shift is computed locally using fast-time samples. Hence, it accounts for local variation of the frequency induced by stochastic fluctuations of speckle.

One can generalize the above defined Kasai estimator to an arbitrary lag, which may be useful when the slow-time samples are composed of beamformed IQ data with different angles. In this case, the Kasai estimator can be expressed straightforwardly as follows:

$$ΔΦ = {∠R}_{m} with {R}_{m} = {\sum }_{k = 1}^{{N}_{EL} - m} {w}_{k + m} {γ}_{k + m} {w}_{k} {γ}_{k}^{*} ,$$

where m accounts for the lag of the correlation.

The parameters for the velocity extraction step are the following:
Ensemble length,
Range gate length (used for Loupas estimator to compute the mean frequency),
Lateral average length (used to filter the correlations along the azimuthal dimension for denoising purpose),
Temporal window coefficients,
Radial window coefficients,
Lateral window coefficients,
Boundary conditions in the different dimensions,
Convolution modes in the different dimensions,
Displacement extraction method: Kasai or Loupas,
Extra lag (in case of lag-m correlation).

### 3.1.2.2. Time-Based Estimators

Time-based estimators (C. Kasai and K. Namekawa, “Real-time two-dimensional blood flow imaging using an autocorrelation technique”, in IEEE 1985 Ultrasonics Symposium, San Francisco, CA, USA, 1985. doi: 10.1109/ultsym.1985.198654) represent an alternative to phase-based estimators suitable for wideband US pulses and large displacements where aliasing of phase-based estimators may occur.

Consider two slow-time two samples γₖ(r) and γₖ₊₁(r) of the same radial-line (or equivalently axial line), where the azimuthal (or equivalently the lateral) coordinate has been ignored for clarity. Assume that the displacement of the medium is radial with constant velocity vᵣ. It can be demonstrated that:

$${γ}_{k + 1} ( r ) = {γ}_{k} ( r + {v}_{r} {T}_{P} ) ,$$

where T_{P} accounts for the pulse repetition period.

Hence, slow-time samples are just time-shifted versions of each other and the time shift depends on the radial velocity. In order to estimate the time-shift, a windowed cross correlation is used such that:

$$C ( r , Δ r ) = {{\int }_{r}}^{r + R} {γ}_{k} ( {r}^{′} ) {γ}_{k + 1} ( {r}^{′} - Δ r ) {dr}^{′} = {{\int }_{r}}^{r + R} {γ}_{k} ( {r}^{′} ) {γ}_{k} ( {r}^{′} + {v}_{r} {T}_{P} - Δ r ) {dr}^{′} , \text{?}$$

which is maximum when Δr=vᵣT_{P}. Hence, maximizing the cross correlation gives an estimate of the radial velocity.

### 3.1.3. Filtering Techniques of the Velocity Maps

This Section aims at providing scientific details related to the filtering phase of the analysis method according to the invention.

The aim of filtering techniques is to isolate the part of the measured displacement that comes from shear wave propagation from other components (e.g., static deformation, compressional waves, out-of-plane shear waves or reflected waves). They may rely on signal processing techniques (e.g. filtering) as well as on clever acquisition or shear-wave generation processes. Few examples are given hereafter.

In transient elastography, when shear wave generation relies on the probe mechanical vibration, the propagative displacement wave is polluted by static deformation, compressional waves and potential interferences.

Different methods are used to reduce the static deformation (see document titled “PROCEDE D'ANALYSE D'UN MILIEU PERMETTANT DE REDUIRE LES EFFETS D'ARTEFACTS DUS A DES DEFORMATION STATIQUES DANS LE MILIEU” whose filing number is FR2210011).

Symmetric sampling (D. C. Mellema et al., “Probe Oscillation Shear Elastography (PROSE): A High Frame-Rate Method for Two-Dimensional Ultrasound Shear Wave Elastography”, IEEE Trans. Med. Imaging, vol. 35, no. 9, pp. 2098-2106, September 2016) is based on acquiring displacement fields at time instants corresponding to the exact same realization of the static deformation.

Impulse vibration generation (S. Catheline, F. Wu, and M. Fink, “A solution to diffraction biases in sonoelasticity: the acoustic impulse technique”, J. Acoust. Soc. Am., vol. 105, no. 5, pp. 2941-2950 May 1999) is based on the generation of a shorter mechanical vibration in order to separate the static deformation from the shear-wave propagation.

Low-pass IIR filtering (D. C. Mellema et al., “Probe Oscillation Shear Elastography (PROSE): A High Frame-Rate Method for Two-Dimensional Ultrasound Shear Wave Elastography”, IEEE Trans. Med. Imaging, vol. 35, no. 9, pp. 2098-2106, September 2016) as well as empirical mode decomposition (D. C. Mellema et al., “Probe Oscillation Shear Wave Elastography: Initial In Vivo Results in Liver”, IEEE Trans. Med. Imaging, vol. 37, no. 5, pp. 1214-1223 May 2018) is a filtering technique that acts on the displacement field directly.

In order to further filter the propagative displacement waves from more common artefacts (e.g., compressional waves, interferences, out-of-plane shear wave reflections), temporal, spatial and spatio-temporal (i.e., directional) filtering techniques exist and are commonly used in nearly all the types of dynamic elastography: magnetic-resonance elastography (A. Manduca, D. S. Lake, S. A. Kruse, and R. L. Ehman, “Spatio-temporal directional filtering for improved inversion of MR elastography images”, Med. Image Anal., vol. 7, no. 4, pp. 465-473, December 2003), time-harmonic elastography (H. Zhao et al., “External vibration multi-directional ultrasound shearwave elastography (EVMUSE): application in liver fibrosis staging”, IEEE Trans. Med. Imaging, vol. 33, no. 11, pp. 2140-2148, November 2014), shear-wave elastography (T. Deffieux, J.-L. Gennisson, J. Bercoff, and M. Tanter, “On the effects of reflected waves in transient shear wave elastography”, IEEE Trans. Ultrason. Ferroelectr. Freq. Control, vol. 58, no. 10, pp. 2032-2035, October 2011). Such methods are based on filtering the displacement field directly.

Temporal filtering is widely used in harmonic elastography as it permits to band-pass filter the displacement field around the frequency of interest. Spatial and spatiotemporal techniques are used in nearly all the elastography methods.

3.1.4. Time-Shift Estimation for Tracking the Shear-Wave Propagation from Velocity Maps

Once the displacements (i.e. the plurality of velocity maps) are computed and filtered, the shear wave velocity is computed from which the stiffness can be inferred. The shear wave velocity, i.e. the group velocity or the velocity of the shear wavefront, can be computed by considering two points in the medium, with known distance and by performing a time-shift estimation between the velocity signals of these two points, along the slow-time scale.

Time-shift estimation from displacements is based on well-known techniques in signal processing. The idea is to compare slow time velocity signals corresponding to two points whose direction is parallel to the shear-wave propagation direction. Such velocity signals are therefore phase-shifted or time-shifted replicas of each other. The time/phase-shift can be recovered using the maximum value of the generalized cross correlation function for instance. Given the distance and the time/phase shift, one can easily deduce the shear wave velocity.

3.1.5. On Transient Elastography with Mechanical Shear Wave Generation
3.1.5.1. Principle and Far-Field Beam Pattern Associated with a Point-Source Excitation

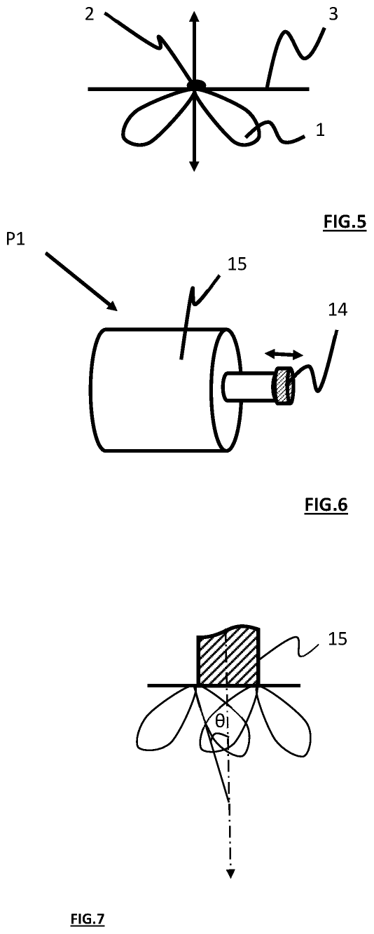

FIG. 5 illustrates a radiation amplitude 1 of a shear wave generated by a point source 2 on the skin 3 of a patient. The point source is for instance a vibrating needle which allows the generation of a shear wave into a patient when the vibrating needle is applied onto the skin of the patient.

The shear wave front is approximately a half sphere centered on an excitation point corresponding to the contact point between the point source and the skin 1 of the patient.

As can be seen by the skilled person, the maximum shear wave amplitude is slanted with the normal from the skin surface, and is unfortunately close to zero at the normal.

### 3.1.5.2. Green Functions of a Point-Source Excitation

The Green function associated with a point-source excitation in an infinite medium can be decomposed into four terms (See S. Catheline and N. Benech, “Longitudinal shear wave and transverse dilatational wave in solids”, J. Acoust. Soc. Am., vol. 137, no. 2, pp. EL200-5, February 2015):

$${G}_{mn} ( 0 , r ) = {G}_{mn}^{S} ( 0 , r ) + {G}_{mn}^{NFS} ( 0 , r ) + {G}_{mn}^{P} ( 0 , r ) + {G}_{mn}^{NFP} ( 0 , r ) ,$$

where m and n account for the directions of the force and the observation and r is the distance from the point source.

$${G}_{mn}^{P} and {G}_{mn}^{S}$$

are the Green functions associated to the well known far-field compressional and shear waves. These waves are polarized longitudinally and transversely, respectively.

The two other Green functions

$${G}_{mn}^{NFS} and {G}_{mn}^{NFP}$$

account for near-field shear wave and compressional wave terms, which have longitudinal and transverse polarization, respectively. Notice that the polarization of the near-field terms is the opposite of the one of the far-field terms. Such terms are near-field terms as they decrease in r⁻².

The predominant terms are the far-field P (compressional) and S (shear) waves. We observe that such terms vanish in directions perpendicular and parallel to the direction of the point force, respectively. Tiny near-field terms can be observed in regions where the far-field terms vanish.

In soft media, compressional waves are negligible such that the Green function reduces to the sum of the two shear-wave terms.

Now, assume that the point force is applied in the vertical direction (n=3) and we observe the field in a direction parallel to the direction of the point force (m=3), we have that

$${G}_{33}^{S} ( z ) = 0 and {G}_{33}^{NFS} ( z ) = \frac{1}{2 πρ {r}^{3}} \frac{{e}^{ikr}}{i ω} ( \frac{r}{β} - \frac{1}{i ω} ) ,$$

where ρ is the density, k accounts for the wavenumber of the shear wave and β is the shear wave velocity.

Hence, in the direction parallel to the direction of the point force, the only remaining propagating term is the near field term which has the same characteristics as the far field term in terms of phase velocity and frequency, but has a longitudinal polarization.

In a semi-infinite medium, i.e. when the point source is located at the surface of the medium, expressions of the Green's functions in the medium are similar to the ones of the infinite medium, except that there is a factor of 2 linked to the fact that the interface plays the role of a mirror of the waves that would propagate in the backward direction (See L. Sandrin, D. Cassereau, and M. Fink, “The role of the coupling term in transient elastography,” J. Acoust. Soc. Am., vol. 115, no. 1, pp. 73-83, January 2004.).

Hence, in a soft medium and in a direction parallel to the direction of the point force, the only remaining term is the shear-wave near field term, which is very weak.

3.1.6. Example of Clinical Systems relying on Transient Elastography for Liver Stiffness Measurement

### 3.1.6.1. Fibroscan®

Fibroscan® system relies on a damped vibration of a mono-element ultrasonic probe. Different probes with various diameters (7 mm, 9 mm, 12 mm) are available depending on the patient (pediatric, adult with different BMI).

With reference to FIG. 6, this probe P1 comprises:
an ultrasonic transducer 14 for the emission of ultrasonic waves and the acquisition of echoes, and
a vibrator 15 forming a non-punctual shear wave source, said vibrator 15 comprising a cylindrical rod whose free end (a disk of a diameter of several millimeters) is adapted to contact the skin of the patient.

The transducer 14 is attached to the end of the vibrator 15. The vibrator 15 allows the transducer 14 to vibrate in order to generate a shear wave. The principle of operation of the medical pulse elastography apparatus is as follows. The vibrator 15 is activated to induce the movement of the transducer 14 and generate a low-frequency shear wave in the tissue to be analyzed. During the propagation of the low-frequency shear wave, the transducer 14 emits and receives high-frequency ultrasonic waves in order to allow the study of the propagation of the low-frequency shear wave.

FIG. 7 illustrates the shear wave amplitude of the shear wave generated by the vibrator 15. As illustrated on FIG. 7, the locus of the shear wave source corresponds to the perimeter of the rod's free end. This is the convolution of the point source green function described before by the ultrasound transducer tip shape.

Shear wave on the axis is no more close to zero because it comes from the additive interference of shear waves coming from the rod periphery with a non-zero angle. Such a phenomenon is due to the fact that the diameter of the rod is non-zero, otherwise only the very weak near field term would have been visible. The larger the diameter of the rod, the larger the angle at a given depth.

If θ is the angle, then the shear wave measured on the axis will have an apparent velocity which is larger than the physical shear wave by a factor

$$\frac{1}{cos θ} .$$

For example, if the rod diameter is 10 mm and the depth of analysis is 40 mm, then the overestimation of the shear wave velocity will be 0.8%, and the overestimation of the stiffness will be 1.6%. This is small, and this is a fixed overestimation linked to the geometry, which can be corrected.

### 3.1.6.2. Hepatoscope

Other types of ultrasonic pulse elastography probe have been proposed in the art. For instance, document WO 2022/084502 describes a probe—called “Hepatoscope”—allowing the measurement of the elasticity of a medium. Referring to FIG. 8, the probe P2 comprises: an inertial vibration exciter 31, a transducer array 32 and other elements (housing, electronic card 33, etc.).

All the elements are mechanically integral except for a mobile part (not shown) of the inertial vibration exciter 31, such that the entire probe vibrates when the inertial vibration exciter 31 is activated.

Such an ultrasonic pulse elastography probe has a more complex shear wave generation diagram. This diagram can be obtained by convoluting the Dirac Stylus diagram of FIG. 5 by the shape of the vibrating probe.

The shear wave intensity generated from every probe surface location will be increasing with the local curvature of the contact surface between the probe and the patient's skin.

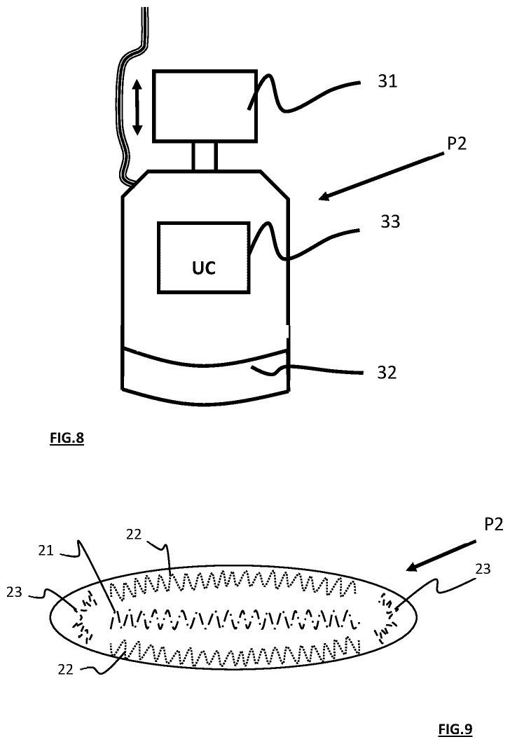

FIG. 9 illustrates a bottom view of the probe P2, with its lens, and the shear wave generating areas:
a first area 21 corresponds to the lens in contact with the patient's skin with a moderate curvature; this first area 21 has moderate shear wave generation capabilities,
a second area 22 corresponds to the longitudinal edges of the contact surface between the probe and the patient's skin; this second area has high shear wave generation capabilities,
a third area 23 corresponds to the lateral edges of the contact surface between the probe and the patient's skin; this third area has fair shear wave generation capabilities.
   3.1.7. Undesirable effects Related to Transient Elastography for Liver Stiffness Measurements

As mentioned above, the previously described types of probes (Fibroscan®, Hepatoscope) have to be positioned into contact with the patient's skin in order to generate a shear wave. When such probes are used to measure viscoelastic properties of an organ such as the patient's liver, said probes are put into contact with a zone of the patient's skin which is located above the patient's ribs.

In such case, different undesirable effects can occur:
a shear wave shading effect on the one hand, and
a mechanical coupling effect on the other hand.

### 3.1.7.1. Shear Wave Shading

Shear wave shading occurs when a “light” force (for instance a force less than 5 Newtons) is applied by a user for contacting the ultrasonic pulse elastography probe onto the patient's skin. This shear wave shading effect is due to the reflection onto the ribs of the shear wave generated by the ultrasonic pulse elastography probe.

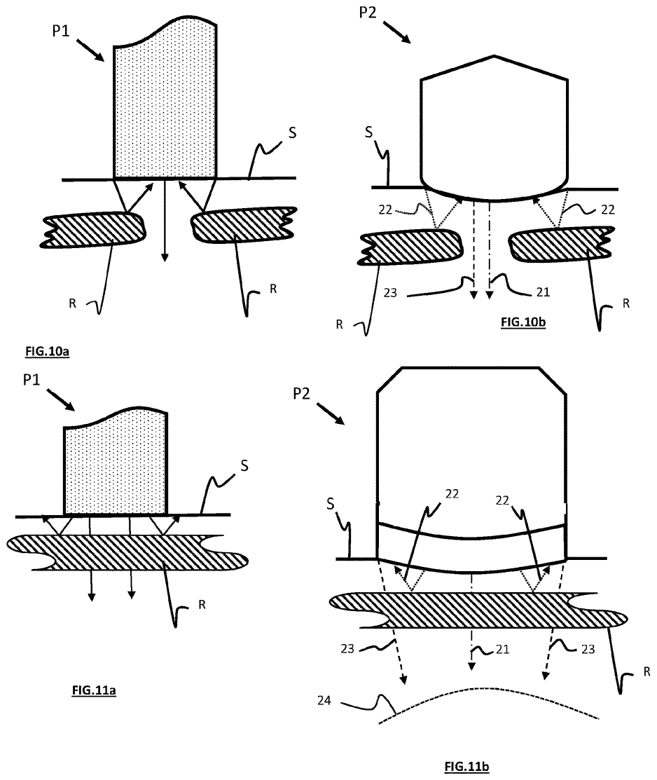

FIGS. 10a and 10b illustrate in a plane perpendicular to the ribs the shear wave shading effect occurring when using a Fibroscan® (FIG. 6) and when using a Hepatoscope (FIG. 8).

As shown on FIG. 10a, in the case of the Fibroscan®, only parts of the rod's free end allow a shear wave to be generated, said shear wave propagating in the medium between two successive ribs. Indeed, the shear wave generated by the portions of the rod's free end which are located above the ribs are shaded and reflected onto the ribs.

As shown on FIG. 10b, in the case of the Hepatoscope, the unique shear waves which are shaded and reflected onto the ribs are the shear waves generated by the second area corresponds to the longitudinal edges of the contact surface between the probe and the patient's skin. The shear waves generated by the first and third areas travel between the ribs and propagate within the medium.

FIGS. 11a and 11b illustrate in a plane parallel to the ribs the propagation of the shear waves within the medium when using a Fibroscan® (FIG. 6) and when using a Hepatoscope (FIG. 8). The skilled person will appreciate that:
in the case of the Fibroscan®, the shear waves propagating through the medium have a low intensity due to the shading (see FIG. 11a). This is the reason why Fibroscan offers different probe sizes in order to minimize the shading effect,
in the case of a Hepatoscope, the shear waves generated by the first and third areas penetrate in the medium below the ribs; the shear wave front resulting from their interference has a concave shape (see FIG. 11b).

### 3.1.7.2. Mechanical Coupling

The mechanical coupling effect occurs when a “high” force (for instance a force greater or equal to 5 Newtons) is applied by a user in order to squeeze the ultrasonic pulse elastography probe against the ribs. In that situation, the probe and ribs displacements are the same, the probe and the ribs move as a single mechanical assembly, and the shear generating areas are limited to the rib's edges and to the lateral edges of the probes in between the ribs.

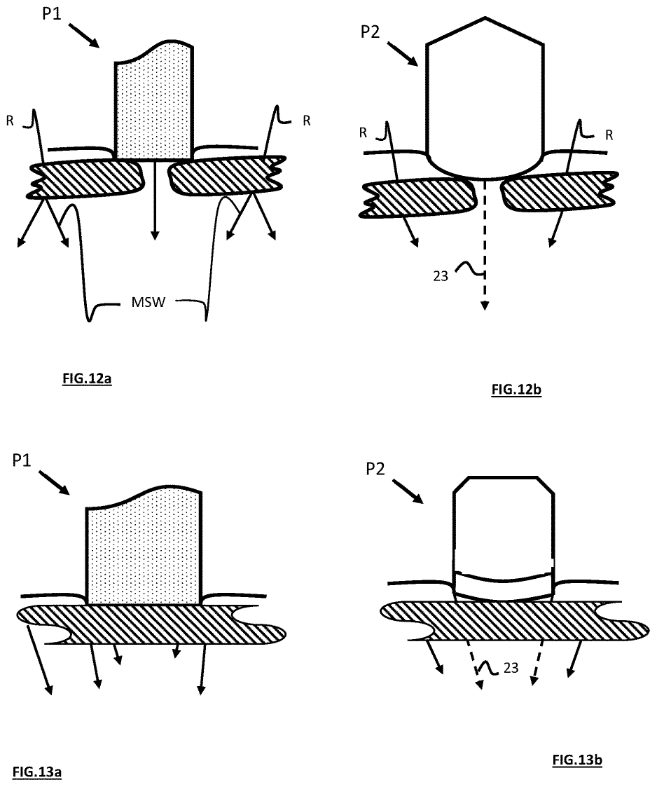

FIGS. 12a, 13a and 12b, 13b illustrate the mechanical coupling effect occurring when using a Fibroscan® (FIG. 12a, 13a) and when using a Hepatoscope (FIG. 12b, 13b), FIGS. 12a and 12b in a plane perpendicular to the ribs and FIGS. 13a and 13b in a plane parallel to the ribs. In both cases the main shear wave generation comes from the outer edge of the ribs, in the region of maximum possible shear between rib and tissue during displacement. No shear wave is generated from probes in the regions where they move along with the tissue. The only remaining faint shear generation from probes are in the region where there is relative displacement against the tissue: at probe edges located between the ribs (third area in the case of the Hepatoscope).

Shear wave generation from ribs is efficient, but create shear wave created by such vibrations have propagation directions:
out of axis for the Fibroscan®, and
out of the imaging plane for Hepatoscope.

Since the geometry (ribs sizes and position) is unknown, the projection angle on the axis or the imaging plane cannot be correct: shear wave velocity estimation will always be overestimated, by an unknown amount.

Regarding Hepatoscope, the angle of the projection of the shear waves fronts on the imaging plane can be determined by processing the plurality of velocity maps. When the probe is symmetrically pressed on two ribs the resulting wave fronts are convex, which allows one to differentiate the case of high pressure (convex wavefront) from the case of low pressure (where the wave fronts are concave).

However, the user may press the probe only on one rib. The induced shear wave front have been analyzed with large angles relative to the normal (above 10 degrees or below-10 degrees).

### 3.1.7.3. Conclusion

When a mechanical coupling effect occurs, it is not possible to derive a quantitative value for the shear wave velocity in the liver. It is thus preferable to avoid (or at least limit) such a mechanical coupling.

In the case of the Fibroscan®, and due to its low efficiency for shear wave generation, it is necessary to impose quite a high pressure between the probe and the patient's skin. To avoid the mechanical coupling effect, it is thus necessary to use a rod's free end having a diameter smaller than the rib spacing. This is the reason why Fibroscan® makes available to users a group of probes (three probes), each probe including a rod's free end having a different diameter. This allows the shear wave intensity to be optimized on the one end, and the shear wave penetration on the other end.

Regarding Hepatoscope where the shear wave generation efficiency is much larger (due to larger mechanical aperture), it is possible to apply a “light” force in order to put the ultrasonic pulse elastography probe in contact with the patient's skin. This limits the mechanical coupling effect, while ensuring that shear waves having a sufficient amplitude are produced and propagate through the medium.

The analysis of shear wave propagation angle in the 2D imaging plane allows one to:
Correct for the bias in stiffness estimation induced by the unknown shear wave propagation within the imaging plane,
Discriminate between geometries specific of light pressure, and geometries specific of rib coupling; this will allow to discard measurements of out of plane rib generated shear waves, and to provide to the user a visual help to avoid this situation.
   3.1.8. Performing 2D Shear Wave Front Angle Analysis from Velocity Signals of Points A, B and C

FIG. 14 illustrates a shear wave front, with propagation velocity V, and three points for analysis in the image: points A, B, and C.

The principle of the invention is to compute the time of flight of the shear wave between:
Point A and point B on a line chosen to be roughly parallel to the propagation direction; Knowing this time of flight allows one to compute the projected velocity V₁ of the shear wave on this line,
Point A and point C on a line chosen to be roughly perpendicular to the propagation direction; Knowing this time of flight allows one to compute the projected velocity V₂ of the shear wave on this line.

Then, from the projected velocities V₁ and V₂, it is possible to estimate a corrected and real propagation velocity V for the shear wave at point A based on the following demonstration.

$$Since : θ = {θ}_{1} + {θ}_{2} , and V = {V}_{1} cos {θ}_{1} = {V}_{2} cos {θ}_{2} ,$$

$$We have that$$

$${V}_{1} cos {θ}_{1} = {V}_{2} cos ( θ - {θ}_{1} ) ⇒ {V}_{1} cos {θ}_{1} = {V}_{2} ( cos θ cos {θ}_{1} + sin θ sin {θ}_{1} ) ⇒ cos {θ}_{1} ( {V}_{1} - {V}_{2} cos θ ) = {V}_{2} sin θ sin {θ}_{1} ⇒ tan {θ}_{1} = \frac{( {V}_{1} - {V}_{2} cos θ )}{{V}_{2} sin θ} ⇒ V = {V}_{1} \sqrt{\frac{1}{1 + {( \frac{( {V}_{1} - {V}_{2} cos θ )}{{V}_{2} sin θ} )}^{2}}}$$

where V₂ is considered positive if the propagative displacement wave travels from A to B and negative if the propagative displacement wave travels from B to A.

If θ is chosen to be close to pi/2, then the expression becomes:

$$V = \frac{{V}_{1} {V}_{2}}{\sqrt{{V}_{1}^{2} + {V}_{2}^{2}}}$$

$$And then$$

$${θ}_{1} = sign ( {V}_{2} ) arccos \frac{V}{{V}_{1}}$$

$$which becomes if θ = \frac{π}{2} :$$

$${θ}_{1} = sign ( {V}_{2} ) arccos \frac{{V}_{2}}{\sqrt{{V}_{1}^{2} + {V}_{2}^{2}}}$$

In a real device, points A and B may be chosen close together on a radial line originating from the convex probe center in order to get a local shear wave velocity, and A and C points might be more distant to get a more robust evaluation of velocity V₂ used for velocity correction.

Hence, the proposed shear wavefront angle analysis allows us to:
unbias the local value of the shear wave velocity, for each point within the medium
obtain local value of the shear wavefront angle using the angle θ₁, and the corresponding azimuthal coordinate of the scan line between A and B.

### 3.1.9. Using Shear Wave Front Angle as Quality Factor, and as an Indicator of Probe Pressure on Tissue

It is clear from the above that “light” probe pressure on the tissue is necessary to avoid the generation of off imaging plane shear waves from the rib edges (due to the mechanical coupling effect).

One possibility, not excluding others, to ensure such a condition is the following:
in order to distinguish between “light” pressure and “high” pressure (which induces rib coupling) we can take advantage on the fact that:
   curvature (concave-convex) of the shear wave front is an indicator of pressure in some cases, especially with the same pressure on top and bottom ribs;
   If the absolute value of the mean shear wave front angle (taken over the ROI) is high (above e.g., 10 degrees), then pressure is probably “high” on one rib (dissymmetric pressure on top and bottom ribs),
we can then conduct the analysis using a ROI which is either on the right or on the left of the probe center and decide for example that:
   if the average shear wave angle in the ROI is in a certain range (e.g., between 0 and −10 degrees, depending on angle sign convention), then pressure is probably ok (“light” pressure, no rib coupling),
   if the average angle is out of this range, then pressure is probably too “high”.

The skilled person will note that this indicator can be combined with other quality factors, such as:
Probe excursion that can be monitored from displacement before shear wave extraction, and/or
Shear wave amplitude.

The skilled person will have understood that:
the quality factor relative to the orientation and position of the probe,
the coefficient representative of the pressure applied by the probe onto the patient, and
the parameter relative to the quality of contact between the probe and the patient, described in this document can be combined in any fashion in order to obtain a compounded quality factor, and/or indicator.

The skilled person will have understood that many modifications may be provided to the invention described earlier without materially departing from the new teachings and advantages described here. Therefore, all the modifications of this type are intended to be incorporated inside the scope of the appended claims.

1. A method of determining 2D characteristics of a propagative displacement wave inside a patient, said method comprising:
estimating a plurality of velocity maps of a target region over time, said estimating phase including the steps consisting in:
constructing a plurality of echogenicity maps by repeatedly implementing the following sub-steps for each echogenicity map:
transmitting ultrasounds signals using an array of transducer elements including a group of transducer elements,
receiving backscattered echo signals by the array of transducer elements, each transducer element allowing the acquisition of a respective temporal signal of a group of temporal signals corresponding to the amplitude of the received backscattered echo signals onto the array of transducer elements,
constructing the echogenicity map from the group of temporal signals,
estimating the plurality of velocity maps by correlating and processing the plurality of echogenicity maps,
filtering the propagative displacement wave within the plurality of velocity maps in order to obtain a filtered plurality of velocity maps,
determining 2D characteristics including the propagation velocity and the propagation direction of the propagative displacement wave for each point of interest A within the plurality of velocity maps in order to obtain at least one map of 2D characteristics, said point of interest A having the same coordinates within the plurality of velocity maps by:
associating at least two points B, C within the plurality of velocity maps such that points A, B, C are non-collinear, said points B, C having the same coordinates within the plurality of velocity images/maps,
extracting a temporal propagative displacement signal from the filtered plurality of velocity maps for each of the three points A, B, C,
estimating longitudinal and transversal apparent times of flight of the propagative displacement wave between points A, B and C using the temporal propagative displacement signals associated to points A, B, C by:
intercorrelating the temporal propagative displacement signals associated to points A, B for estimating the longitudinal apparent time of flight of the propagative displacement wave from point A to B,
intercorrelating the temporal propagative displacement signals associated to points A, C for estimating the transversal apparent time of flight of the propagative displacement wave from point A to C,
determining propagation direction and propagation velocity of the propagative displacement wave based on the transversal and longitudinal apparent times of flight.

2. The method according to claim 1, further comprising a step of estimating a stiffness of a tissue contained within the target region using the map of 2D characteristics.

3. The method according to claim 2, wherein the step of estimating the stiffness of the tissue includes:
extracting velocities of the propagative displacement wave for a subset of points from the map of 2D characteristic, said subset of points being representative of the tissue contained within the target region, and
computing a median, or an average of extracted velocities of the propagative displacement wave for the subset of points, and
estimating the stiffness from said computed median or average.

4. The method according to claim 2, wherein the step of estimating the stiffness of the tissue includes:
computing a map of stiffness of the region of interest from the map of 2D characteristics,
extracting stiffnesses of a subset of points from the map of stiffness, said subset of points being representative of the tissue contained within the target region, and
computing a median, or an average of extracted stiffnesses of the subset of points.

5. The method according to claim 1, further comprising a step of determining a quality factor relative to an average propagative displacement direction, said step of determining the quality factor including:
extracting directions of the propagative displacement wave for a subset of points from the map of 2D characteristics,
computing an average direction of propagation of the propagative displacement wave from the extracted directions of the propagative displacement wave for the subset of points, and
comparing the average direction of propagation of the propagative displacement wave to a predefined range of directions to obtain a comparative data, and
deriving the quality factor according to the comparative data.

6. The method according to claim 1, further comprising a step of determining a coefficient representative of the pressure applied by the array of transducers (T₁-Tₙ) onto the patient, said step of determining the coefficient including the sub-steps of:
computing a curvature of the wave front of the propagative displacement wave based on the map of 2D characteristics,
if the computed curvature is concave corresponding to a convergent propagative displacement wave, assigning to the coefficient a value representative of a light pressure,
if the computed curvature is convex corresponding to a diverging propagative displacement wave, assign to the coefficient a value representative of a strong pressure higher than the light pressure.

7. The method according to claim 1, further comprising a step of determining a parameter representative of the quality of the propagative 2D displacement estimation, said step of determining the parameter including the sub-steps of:
computing a variance of the transversal apparent time of flight which is representative a spatial variance of the direction of the propagative displacement wave,
comparing said quantity to a predefined threshold and determining the parameter according to the result of said comparison sub-step.

8. The method according to claim 1, wherein the step of estimating the plurality of velocity images/maps by correlating and processing the plurality of complex echogenicity maps includes:
cross-correlating the plurality of complex echogenicity maps pair by pair with a certain temporal lag to obtain a plurality of phase-shift maps,
deriving the plurality of velocity maps from related plurality of phase-shift maps.

9. The method according to claim 1, wherein the sub-step of constructing the echogenicity map from the group of temporal signals comprises the resolution of an inverse problem to produce said echogenicity map.
